(19) United States
(12) Patent Application Publication
(10) Pub. No.: US 2026/0305990 A1
(43) Pub. Date: Oct. 8, 2026

(54) USE OF AN OPTICAL EMITTER IN AN IMPLANTABLE MEDICAL DEVICE TO TRANSMIT INFORMATION TO AN OPERATOR OR PATIENT

(71) Applicant: Medtronic, Inc., Minneapolis, MN (US)

(72) Inventors: Catherine R. Condie, Shoreview, MN (US); Ryan D. Wyszynski, Oak Grove, MN (US); Anna J. Malin, Paradise Valley, AZ (US); Nicholas H. Finstrom, Coon Rapids, MN (US)

(21) Appl. No.: 19/479,133

(22) PCT Filed: Apr. 1, 2024
(86) PCT No.: PCT/IB2024/053150

§ 371 Date: Oct. 27, 2025

Related U.S. Application Data
(60) Provisional application No. 63/498,318, filed on Apr. 26, 2023.

(51) Int. Cl. A61B 5/1455; A61B 5/00

(52) U.S. Cl. CPC A61B 5/1455; A61B 5/0031; A61B 5/686; A61B 2562/04

(57) ABSTRACT

An implantable medical device (IMD) comprises a housing and a plurality of sensors on or within the housing. The housing is configured for subcutaneous implantation within a patient. The plurality of sensors are configured to sense one or more physiological parameters of the patient. The plurality of sensors comprise an optical sensor comprising an optical emitter and an optical detector. Processing circuitry of the IMD within the housing is configured to determine values of the one more physiological parameters using the plurality of sensors, and is configured to determine an operational status of the IMD, and cause the optical emitter to provide a visible indication of the operational status of the IMD.

### FIELD

This application claims the benefit of U.S. Provisional Patent Application Ser. No. 63/498,318, filed Apr. 26, 2023, the entire content of which is incorporated herein by reference.

### FIELD

The disclosure relates generally to medical devices and, more particularly, implantable medical devices configured to monitor patient activity for changes in patient health.

### BACKGROUND

Implantable (insertable) cardiac monitors (ICM) are one category of implantable medical devices (IMD) used for monitoring a patient's heart. ICMs can be inserted under the patient's skin and can monitor and record cardiac data for several years. Implanting an ICM is a relatively simple procedure involving a small incision, often in the patient's chest, and insertion of the ICM into the incision.

### SUMMARY

In various aspects, the present disclosure provides an implantable medical device (IMD) having an optical emitter that provides a visible indication of an operational status of the IMD. The optical emitter may be coupled with an optical sensor and configured for physiological parameter sensing, e.g., photoplethysmography (PPG) or pulse oximetry. The light from the emitter can be utilized to provide a visible indication of an operational status of the IMD. For example, using a suitable blink timing, pattern, and/or color, a light may indicate that the IMD is in a fully operational state and is ready for implantation. The light may further guide the insertion procedure, letting the practitioner know when the IMD detects it has been inserted and when the IMD detects that it is adequately sensing patient parameters.

In some examples, the light may be visible through a patient's skin such that the visible indication of the operational status of the IMD can be seen and used post-implantation. In some examples, the IMD may further include an accelerometer or other suitable sensor for sensing a tapping of the device (either directly or through the patient's skin). With this accelerometer and light combination, the IMD can have both an input and output function for a patient, someone helping a patient, or a clinician. For example, a suitable tapping of the IMD in the patient's chest may signal to the device to run a certain test or scan, to change into a different mode, to store cardiac data relating to a contemporaneous medical symptom, etc. Different tapping patterns or timing may be used to signal different commands to the IMD. And in response, the light can inform that a test or scan is being performed or is complete, that the IMD has changed into a different mode, that data is being stored and/or sent to a clinician, etc.

When an IMD includes an optical emitter as described herein, an implantation procedure can be simplified and streamlined, reducing the cost of the procedure and the patient discomfort that the procedure may cause. For example, current IMD implantation or other interactions may necessitate use an external programmer or other computing device to communicate with the IMD to turn it on after it has been removed from its packaging, to confirm its operational status being ready for insertion, and to confirm that the device has been inserted properly. By using an optical emitter as described herein, a clinician may reduce or eliminate the use of such a programmer device during the procedure. That is, the light may provide a visible indication that an IMD has been removed from its packaging and is ready for implantation, and that an IMD has been inserted properly. Thus, by reducing the need for external instruments the implantation procedure can be less complex, potentially allowing more clinicians to perform the procedure.

In one example, an implantable medical device (IMD) comprises: a housing configured for subcutaneous implantation within a patient; a plurality of sensors on or within the housing, the plurality of sensors configured to sense one or more physiological parameters of the patient, wherein the plurality of sensors comprise an optical sensor comprising an optical emitter and an optical detector; and processing circuitry within the housing and configured to determine values of the one more physiological parameters using the plurality of sensors. The processing circuitry is configured to: determine an operational status of the IMD; and cause the optical emitter to provide a visible indication of the operational status of the IMD.

In another example, a method of controlling an implantable medical device (IMD) to provide a visible indication to a user comprises: determining, by processing circuitry of the IMD, an operational status of the IMD, wherein the IMD further comprises a housing configured for subcutaneous implantation within a patient and a plurality of sensors on or within the housing, the plurality of sensors configured to sense one or more physiological parameters of the patient, wherein the plurality of sensors comprise an optical sensor comprising an optical emitter and an optical detector; and causing, by the processing circuitry, the optical emitter to provide a visible indication of the operational status of the IMD.

In another example, a non-transitory computer readable storage medium comprises program instructions that cause processing circuitry of an implantable medical device (IMD) to perform a method comprising: determining an operational status of the IMD, wherein the IMD further comprises a housing configured for subcutaneous implantation within a patient and a plurality of sensors on or within the housing, the plurality of sensors configured to sense one or more physiological parameters of the patient, wherein the plurality of sensors comprise an optical sensor comprising an optical emitter and an optical detector; and causing the optical emitter to provide a visible indication of the operational status of the IMD.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a block diagram illustrating components of an IMD according to one or more examples of the present disclosure.

FIG. 3 is a perspective drawing illustrating an example configuration of an IMD according to one or more examples of the present disclosure.

FIG. 4 is a flow chart illustrating an example of a process for programmer-less IMD implantation according to one or more examples of the present disclosure.

FIG. 5 is a flow chart illustrating an example of a process for implantation detection according to one or more examples of the present disclosure.

FIG. 6 is a flow chart illustrating an example of a process for detecting tapping the IMD according to one or more examples of the present disclosure.

Like reference characters denote like elements throughout the description and figures.

### DETAILED DESCRIPTION

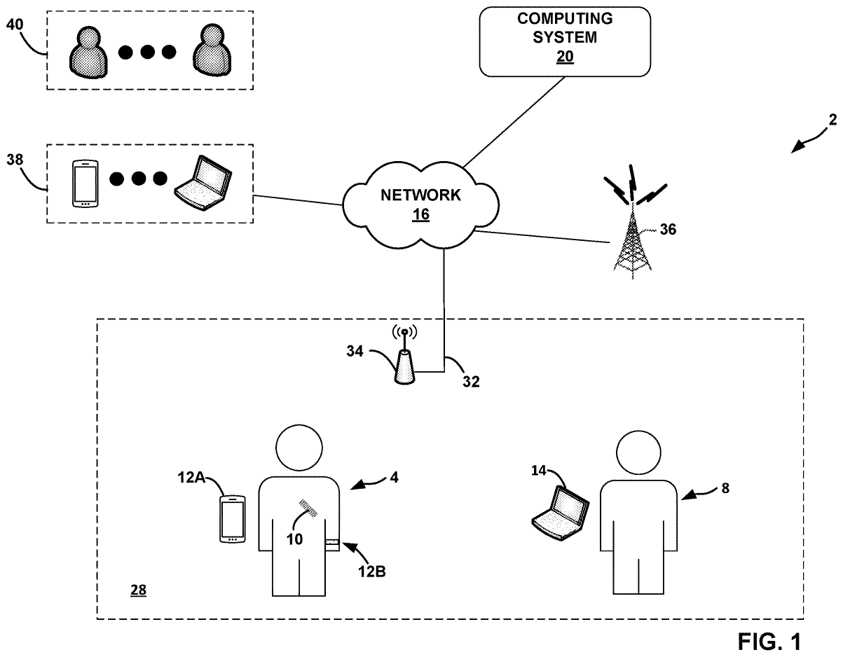

FIG. 1 is a block diagram illustrating an example medical system 2 including an IMD 10 in conjunction with a patient 4, according to some aspects of this disclosure. IMD 10 is configured for continuous, long-term monitoring of the heart of patient 4. For example, IMD 10 may include a plurality of sensors and be configured to continuously (e.g., periodically or on a triggered basis within user intervention) sense one or more physiological parameters using the plurality of sensors.

As examples, IMD 10 may be a pacemaker or implantable cardioverter-defibrillator, which may be coupled to intravascular or extravascular leads, or a pacemaker with a housing configured for implantation within the heart, which may be leadless. Some IMDs do not provide therapy, such as implantable patient monitors. IMD 10 may be such an IMD, e.g., the Reveal LINQ™ or LINQ II™ Insertable Cardiac Monitor (ICM), available from Medtronic, Inc., of Minneapolis, Minnesota, which may be inserted subcutaneously. Such IMDs may facilitate relatively longer-term and continuous monitoring of patients during normal daily activities, and may periodically transmit collected data to a remote patient monitoring system, such as the Medtronic Carelink™ Network.

IMD 10 may determine values of physiological parameters, e.g., based on physiological signals sensed by the IMD using the plurality of sensors. The patient parameters may include, as examples, fluid level, heart rate, respiration rate, patient activity, temperature, heart sounds, oxygenation, and R-wave morphological characteristics. Other example patient metrics include coughing, speech, motion/activity, posture, tissue perfusion, hematocrit, thoracic impedance, subcutaneous impedance, intracardiac impedance, heart rate variability (HRV), weight, blood pressure, sleep apnea burden (which may be derived from respiration rate), ischemia burden, sleep duration, sleep quality, PVC burden, the occurrence, frequency or duration cardiac arrhythmias or other events, and sensed cardiac intervals (e.g., Q-T intervals). Another example patient metric is the ventricular rate during AF. The concentration or levels of various substances, such as blood glucose, hematocrit, troponin and/or brain natriuretic peptide (BNP) levels, within the patient may also be used as one or more patient metrics.

In addition to IMD 10, system 2 includes one or more patient computing devices, e.g., patient computing devices 12A and 12B (collectively, “patient computing devices 12”). Patient computing device(s) 12 are configured for wireless communication with IMD 10. Computing device(s) 12 retrieve parameter data from IMD 10. In some examples, computing device(s) 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing device(s) 12 may communicate with IMD 10 and each other according to the Bluetooth® or Bluetooth® Low Energy (BLE) protocols, as examples. In some examples, only one of computing device(s) 12, e.g., computing device 12A, is configured for communication with IMD 10, e.g., due to execution of software (e.g., part of a health monitoring application as described herein) enabling communication and interaction with an IMD. In some examples, computing device 12B is a smartwatch or other accessory or peripheral for a smartphone computing device 12A.

One or more of computing device(s) 12 may be configured to communicate with a variety of other devices or systems via a network 16. For example, one or more of computing device(s) 12 may be configured to communicate with one or more computing systems, e.g., computing system 20, via network 16. Computing system 20 may be managed by a manufacturer of IMD 10 to, for example, provide cloud storage and analysis of collected data, maintenance and software services, or other networked functionality for their respective devices and users thereof. Computing system 20 may comprise, or may be implemented by, the Medtronic Carelink™ Network, in some examples. Computing device(s) 12 may transmit data, including data retrieved from IMD 10, to computing system 20 via network 16. The data may include sensed data, e.g., values of physiological parameters measured by IMD 10 and other physiological signals or data recorded by IMD 10 and/or computing device(s) 12.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted. In some examples, IMD 10 may be configured to transmit such data to wireless access point 34 and/or computing device(s) 12. Wireless access points 34 and/or computing device(s) 12 may then communicate the retrieved data to computing systems 20 via network 16.

In some cases, computing system 20 may be configured to provide a secure storage site for data that has been collected from IMD 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians 40, via clinician computing devices 38. One or more aspects of the example system described with reference to FIG. 1 may be implemented with general network technology and functionality, which may be similar to that provided by the Medtronic CareLink® Network. In some examples, one or more of clinician computing devices 38 may be a tablet or other smart device located with a clinician, by which the clinician may program, receive alerts from, and/or interrogate IMD 10. For example, the clinician may access data collected by IMD 10 through a clinician computing device 38, such as when patient 4 is in between clinician visits, to check on a status of a medical condition.

Environment 28 includes computing facilities, e.g., a local network 32, by which computing device(s) 12, IoT device 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access point 34 that provides support for wireless communications throughout environment 28. Additionally or alternatively, e.g., when local network is unavailable, computing device(s) 12, IoT devices 30, and other devices within environment 28 may be configured to communicate with network 16, e.g., with HMS 22, via a cellular base station 36 and a cellular network.

As illustrated in FIG. 1, an operator 8, e.g., a clinician or caregiver for patient 4, is in environment 28 with patient 4. Environment may be hospital or clinic, e.g., during a procedure for implanting IMD 10 or a follow-up visit, or may be a residence or long-term care facility of patient 4. Operator 8 is shown with a computing device 14. In some examples, computing device 14 may be a programmer device used by a clinician for activating and configuring the IMD 10 before implantation.

As discussed further below, an IMD 10 configured according to the present disclosure includes an optical emitter of an optical sensor. IMD 10 can provide visible indications to a user, e.g., operator 8 and/or patient 4, via the optical emitter, e.g., during an implantation procedure or after IMD 10 is implanted and closed within patient 4. IMD 10 configured to operate according to the techniques of this disclosure can provide for programmer-less implantation procedures, or implantation procedures less reliant on a programmer. Further, IMD 10 configured according to the present disclosure can allow for command input/output to/from the IMD 10 without need for computing device 14.

Although described in the context of examples in which IMD 10 that senses patient cardiac activity may comprise an ICM, example systems including one or more implantable, wearable, or external devices of any type configured to sense physiological parameters of a patient may be configured to implement the techniques of this disclosure.

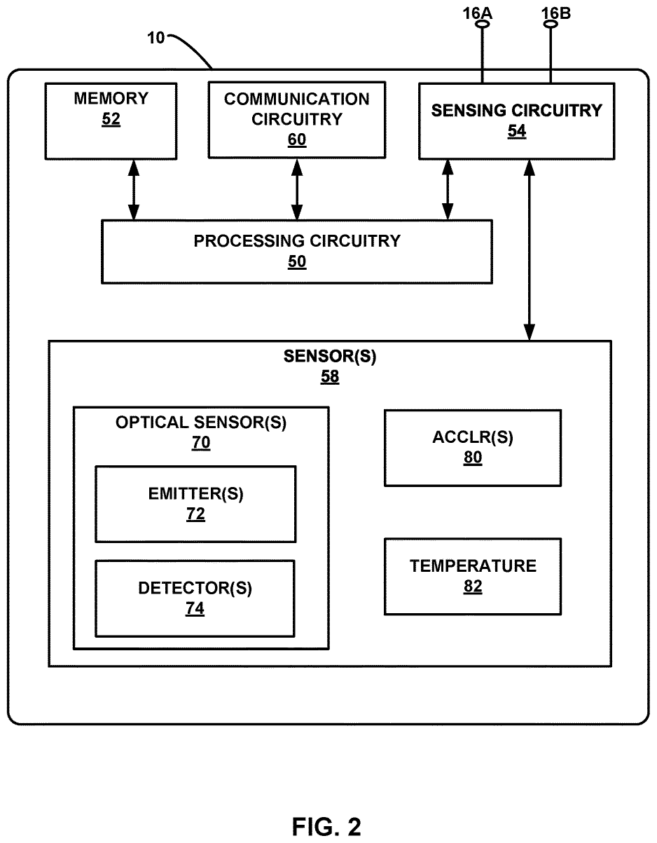

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 16A and 16B (hereinafter, “electrodes 116”) and one or more additional sensor(s) 58, and communication circuitry 60.

Processing circuitry 50 may include fixed function circuitry and/or programmable processing circuitry. Processing circuitry 50 may include any one or more of a microprocessor, a controller, a GPU, a TPU, a DSP, an ASIC, a FPGA, or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 52 includes computer-readable instructions that, when executed by processing circuitry 50, cause IMD 10 and processing circuitry 50 to perform various functions attributed herein to IMD 10 and processing circuitry 50. Memory 152 may include any volatile, non-volatile, magnetic, optical, or electrical media, such as a RAM, ROM, NVRAM, EEPROM, flash memory, or any other digital media.

Sensing circuitry 54 may sense an ECG and measure impedance, e.g., of tissue proximate to IMD 10, via electrodes 16. The measured impedance may vary based on respiration, cardiac pulse or flow, and a degree of perfusion or edema. Processing circuitry 50 may determine patient metrics relating to respiration, fluid retention, cardiac pulse or flow, perfusion, and/or edema based on the measured impedance. In some examples, processing circuitry 50 may identify features of the sensed ECG, such as heart rate, heart rate variability, T-wave alternans, intra-beat intervals (e.g., QT intervals), and/or ECG morphologic features.

In some examples, IMD 10 includes one or more sensors 58 in addition to electrodes 16, such as one or more optical sensors 70, accelerometers 80, temperature sensors 82, microphones, pressure sensors, and/or chemical sensors. In some examples, sensing circuitry 52 may include one or more filters and amplifiers for filtering and amplifying signals received from one or more of electrodes 16 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological parameter data, e.g., values of physiological parameters of patient 4, based on signals from electrodes 16 and sensors 58, which may be stored as data in memory 52.

Processing circuitry 50 may communicate the parameter data to one or more other computing devices, e. g .., computing device(s) 12, 14, and/or computing system 20, using communication circuitry 60. Communication circuitry 60 may include any suitable hardware, firmware, software or any combination thereof for wirelessly communicating with another device. Communication circuitry 60 may be configured to transmit and/or receive signals via inductive coupling, electromagnetic coupling, Near Field Communication (NFC), Radio Frequency (RF) communication, Bluetooth®, WiFi, or other proprietary or non-proprietary wireless communication schemes.

As illustrated in FIG. 2, each of one or more optical sensors 70 may include one or more optical (light) emitters 72 and one or more optical detectors (or photodetectors) 74. Processing circuitry 50 may user optical sensor(s) 70 for one or both of photoplethysmography (PPG) or pulse oximetry sensor. According to the techniques of this disclosure, processing circuitry 50 may control one or more optical emitters 72 to provide a visible indication to a user, and thereby communicate information to the user. In some examples, processing circuitry 50 determines an operational status of IMD 10, and responsively controls optical emitter(s) 72 to provide a visible indication of the operational status.

In various aspects, the optical emitter(s) 72 may be configured to provide a visible indication of the operational status of the IMD, or communicating other information, by varying a blink timing or pattern, color, or brightness of the light it emits. Operation of IMD 10 including optical emitter(s) 72 as described herein provides for several advantages. For example, such operation provides a mechanism to obtain information about IMD 10 for a clinician during an implantation procedure, and for a patient, their caregiver, or a clinician after implantation of the IMD 10. For example, a clinician may be enabled to perform an implantation procedure of the IMD 10 without needing to use a programmer or other external device to determine to the operational status of the IMD 10. This can make the implantation procedure less complex, potentially expanding the set of clinicians that can perform an implantation procedure and making such an IMD more readily available to patients.

In a further aspect, processing circuitry and optical emitter(s) 72 may be configured to communicate information via a suitable serial signaling protocol (e.g., the joint test action group (JTAG) protocol). For example, a blinking light (either before implantation, post-implantation through a patient's skin, or both) can be decoded by using a suitably programmed computing device 12,14 with a camera. By the same token, processing circuitry 509 may receive information from computing device 12,14 having a camera flash or other light emitter via optical detector 74 using the same or similar JTAG or other serial signaling protocol.

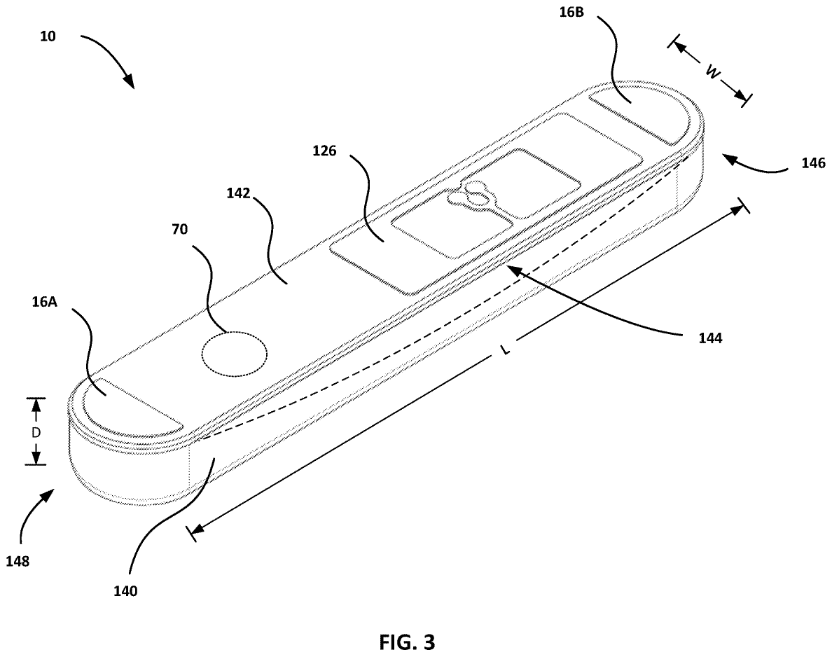

FIG. 3 is a perspective drawing illustrating an example configuration of IMD 10. IMD 10 may include a leadless, subcutaneously-implantable monitoring device, e.g. an ICM. IMD 10 includes housing having a base 140 and an insulative cover 142. Proximal electrode 16A and distal electrode 16B may be formed or placed on an outer surface of cover 142. Various circuitries and components of IMD 10, e.g., described with respect to FIG. 2, may be formed or placed on an inner surface of cover 142, or within base 140. In some examples, a battery or other power source of IMD 10 may be included within base 140. In the illustrated example, antenna 126 is formed or placed on the outer surface of cover 142, but may be formed or placed on the inner surface in some examples. In some examples, insulative cover 142 may be positioned over an open base 140 such that base 140 and cover 142 enclose the circuitries and other components and protect them from fluids such as body fluids. The housing including base 140 and insulative cover 142 may be hermetically sealed and configured for subcutaneous implantation.

Circuitries and components may be formed on the inner side of insulative cover 142, such as by using flip-chip technology. Insulative cover 142 may be flipped onto a base 140. When flipped and placed onto base 140, the components of IMD 10 formed on the inner side of insulative cover 142 may be positioned in a gap 144 defined by base 140. Electrodes 16A and 16B and antenna 126 may be electrically connected to circuitry formed on the inner side of insulative cover 142, e.g., sensing circuitry 54 and communication circuitry 60 as discussed with respect to FIG. 2, through one or more vias (not shown) formed through insulative cover 142. Insulative cover 142 may be formed of sapphire (i.e., corundum), glass, parylene, and/or any other suitable insulating material. Base 140 may be formed from titanium or any other suitable material (e.g., a biocompatible material). Electrodes 16A and 16B may be formed from any of stainless steel, titanium, platinum, iridium, or alloys thereof. In addition, electrodes 16A and 16B may be coated with a material such as titanium nitride or fractal titanium nitride, although other suitable materials and coatings for such electrodes may be used.

In the example shown in FIG. 3, the housing of IMD 10 defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 116C and distal electrode 116D may range from 5 mm to 50 mm, from 30 mm to 50 mm, from 35 mm to 45 mm, and may be any single spacing or range of spacings from 5 mm to 50 mm, such as approximately 40 mm. In addition, IMD 10 may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10 may range from 2 mm to 15 mm, from 5 mm to 15 mm, or from 3 mm to 5 mm, and may be any single depth or range of depths between 2 mm and 15 mm, such as approximately 4 mm. IMD 10 may have a volume of three cubic centimeters (cm) or less, or 1.5 cubic cm or less, such as approximately 1.4 cubic cm.

In the example shown in FIG. 3, once inserted subcutaneously within the patient, outer surface of cover 142 faces outward, toward the skin of the patient. In addition, as shown in FIG. 3, proximal end 146 and distal end 148 are rounded to reduce discomfort and irritation to surrounding tissue once inserted under the skin of the patient. In addition, edges of IMD 10 may be rounded.

FIG. 3 conceptually illustrates optical sensor 70 of IMD 10. Optical sensor 70 may be formed on an inner or outer surface of cover 142, or otherwise under cover within gap 144. As illustrated in FIG. 3, optical sensor 70 (including optical emitter(s) 72 and optical detector(s) 74) may be located along length L of IMD 10 between electrodes 16A and 16B. Since cover 142 faces outward relative to patient 4 (toward the patient's skin) when IMD 10 is implanted, optical emitter(s) 72 and optical detector(s) 74 may also face outward relative to patient 4. Cover 142 may be transparent or translucent so that one or both of optical emitter(s) 72 and optical detector(s) 74 may be beneath cover 142 but still emit and detect light outward relative to patient 4. Consequently, light emitted by optical emitter(s) 72 may be observed by a user of the IMD 10 in an implantation pocket and, in some cases, through the skin when the incision to implant IMD 10 is closed. IMD 10, including instrument and method for inserting IMD 10 is described, for example, in U.S. Patent Publication No. 2014/0276928, incorporated herein by reference in its entirety.

In some examples of the techniques of this disclosure, processing circuitry 50 may determine an operational status of IMD 10, and cause optical emitter(s) 72 to provide a visible indication of the operational status of the IMD. The operational status may correspond to at least one of IMD 10 being removed from packaging or ready for implantation. Processing circuitry 50 may detect removal of IMD 10 from its packaging via electrodes 16 and/or other sensors 58, such as optical sensors and/or accelerometer(s) 80. For example, packaging for IMD 10 may be configured to mask optical detector(s) 74 and/or provide contacts for electrodes 16 that present an impedance to sensing circuitry 54. Processing circuitry 50 may be configured to determine that IMD 10 has been removed from packaging by at least detecting light with optical detector(s) 74 and/or detecting a change in impedance via sensing circuitry 54 and electrodes 16.

Processing circuitry 50 may power on or up circuitry of IMD 10 in response to determining that IMD 10 has been removed from packaging, e.g., communication circuitry 60, sensing circuitry 54, and/or sensor(s) 58. For example, processing circuitry 50 may control sensing circuitry 54 and/or sensor(s) 58 to detect when IMD 10 is implanted within patient in response to detecting that IMD 10 has been removed from packaging. As another example, processing circuitry 50 may activate a communication mode of the communication circuitry 60 based on determining that IMD 10 has been removed from packaging. The communication mode may be an active mode in which IMD 10 is available for and/or tries to establish communication with a computing device, e.g., computing device 14 of an implanting clinician. Processing circuitry 50 may control communication circuitry 60 to begin advertising for Bluetooth® Low Energy session with the computing device in response to determining that IMD 10 has been removed from packaging and/or is ready for implantation. In this manner, communication between IMD 10 and the computing device, e.g., provision of a real-time ECG from IMD 10 to the computing device, may be expedited. Processing circuitry 50 may control optical emitter(s) 72 to provide one or more visible indications indicating that circuitry has been activated in response to detecting removal of IMD 10 from packaging, that communication circuitry 60 has begun advertising for connection, and/or that a connection has been established by communication circuitry 60.

In some examples, the operational status corresponds to IMD 10 having been implanted into the patient. Processing circuitry 50 may detect implantation of IMD 10 based on one or more signals from one or more of electrodes 16 and/or other sensors 58. For example, processing circuitry 50 may detect an ECG, respiration, or impedance via electrodes, PPG or oxygenation signals via optical sensor 70, temperature via temperature sensor 82, or certain motion/orientation via accelerometer 80. Processing circuitry 50 may activate a communication mode of the communication circuitry, e.g., control the communication circuitry to start advertising for a connection with a computing device, based on determining that the IMD has been implanted within the patient. Processing circuitry 50 may control optical emitter(s) 72 to provide one or more visible indications indicating that circuitry or functionality of IMD 10 has been activated in response to detecting implantation of IMD 10, that communication circuitry 60 has begun advertising for connection, and/or that a connection has been established by communication circuitry 60.

In some examples, the operational status corresponds to a quality of measurement of at least one physiological parameter of the one or more physiological parameters by at least one sensor of the plurality of sensors. Processing circuitry 50 may evaluate measurement quality after, e.g., in response to, determining that IMD 10 is implanted withing patient 4. To determine the quality of measurement, processing circuitry 50 may be configured to compare a metric of a signal from the at least one sensor, e.g., at least one of electrodes 16 and/or sensors 58, to a threshold. Processing circuitry 50 may control optical emitter(s) 72 to provide one or more visible indications indicating that signal quality is adequate, and may provide multiple, and in some cases different, visible indication for different sensor signals. In some examples, the operational status corresponds to processing circuitry 50 detecting at least one of a cardiac depolarization or a cardiac contraction via at least one sensor. In some examples, processing circuitry 50 may control optical emitter(s) 72 to output a respective visible indication in response to detection of each of a plurality of cardiac depolarizations or cardiac contractions. In this manner, the user may visualize the detection of the heart rate of patient 4 and, in some cases, compare the cardiac sensing by IMD 10 to an ECG sensed via an external device, e.g., used during the implantation procedure or other clinic visit.

In some examples, processing circuitry 50 may control optical emitter(s) 72 to provide a visual indication to a user, e.g., patient 4 or a caregiver, on the status of IMD 10—either scheduled or in response to an alert condition. In some examples, processing circuitry 50 may control optical emitter(s) 72 to provide a visual indication to a user on the status of a measurement of interest to the user, e.g., indicating that a measurement was made in response to a symptom marking input from patient 4. In some examples, patient 4 or another user may interact with IMD 10 may tapping on IMD 10 while implanted in patient 4. Processing circuitry 50 may detect this user input via accelerometer(s) 80, and responsively activate optical emitter(s) 72, e.g., to indicate receipt of the tapping input and/or to indicate that the action requested via the tapping input was undertaken by IMD 10.

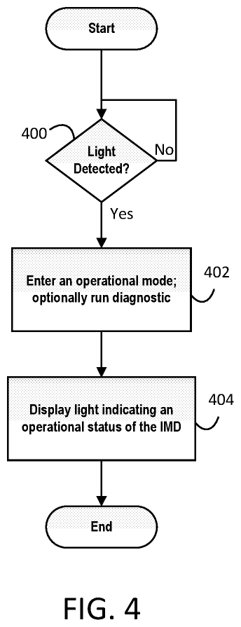

FIG. 4 is a flow chart illustrating an example process for an IMD 10 according to some aspects of this disclosure.

During manufacturing or packing of the IMD 10, a mask (e.g., a sticker) may be applied to the optical sensor 70, e.g., to optical detector(s) 74, or optical sensor 70 may otherwise be obscured (e.g., where the IMD 10 is placed inside an opaque packaging material). The IMD 10 may be placed in a standby or shipping mode, where one or more peripheral circuits may be shut off or depowered, such as the communication circuitry 60, accelerometer 80, or ECG or impedance monitoring using sensing circuitry 54. In this way, the IMD 10 may save power consumed while the IMD 10 is packaged and before an implantation procedure. Optical sensor 70, e.g., optical detector(s) 74, may remain in an operational state, such that removal of the IMD 10 from its packaging and/or removal of a mask from the optical sensor 70 can be detected by sensing light entering the optical detector(s) 74 (Yes of 400). So long is optical detector(s) 74 do not detect light (No of 400), IMD 10 may remain in this standby or shipping mode.

When the optical detector(s) 74 senses light (Yes of 400), it may indicate to processing circuitry 50 that the IMD 10 has been removed from its packaging.

Accordingly, when optical detector(s) 74 sense light, processing circuitry 50 may cause IMD 10 may enter an operational mode (402). For example, processing circuitry 50 may power on or place into an active state one or more peripheral circuits that were in an off state or standby state while the IMD 10 was packaged. Optionally, processing circuitry 50 may run or execute a diagnostic test or perform any other suitable operations to prepare the IMD 10 for implantation. When the IMD 10 is ready for implantation, or if there is an issue with the IMD 10, processing circuitry 50 may cause optical emitter(s) 72 to display a light indicating an operational status of the IMD 10 (404). Using a suitable blink timing or pattern, or color (e.g., green), or brightness, or some combination of the above, the optical emitter(s) may indicate, for example, that the IMD 10 is ready for implantation.

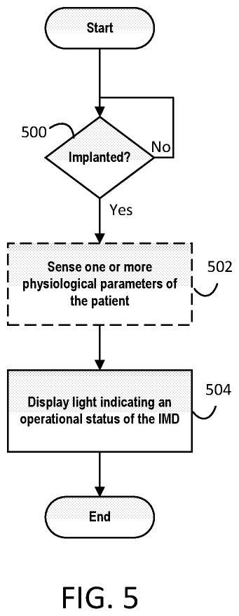

FIG. 5 is a flow chart illustrating another exemplary process for an IMD 10 according to some aspects of this disclosure.

Processing circuitry 50 may detect if/when the IMD 10 has been implanted based on one or more signals from one or more of electrodes 16 and/or sensors 58 (500). For example, sensing circuitry 54 may measure an impedance between electrodes 16, and this impedance may change after the IMD 10 is implanted. Any suitable sensor or combination of sensors capable of detecting whether the IMD 10 has been implanted may be used within the scope of this disclosure.

When processing circuitry 50 detects that IMD 10 has been implanted (Yes of 500), processing circuitry 50 may control sensing circuitry 54 and/or sensors 58 to sense one or more physiological parameters of the patient (502). For example, processing circuitry 50 may control sensing circuitry 54 to measure cardiac electrical activity via electrodes 16 after detecting that IMD 10 has been implanted. Processing circuitry 50 may then control light emitter(s) display a light indicating an operational status of the IMD 10 (504). The operational status may be any suitable status. For example, the operational status may indicate whether the IMD 10 has been implanted properly based on signals from sensing circuitry 54 and/or sensor(s) 58. In addition or alternative, the operational status may indicate whether the quality of measurement of the physiological parameter is acceptable. For example, processing circuitry 50 may control optical emitter(s) 72 to provide visible feedback on whether the sensing circuitry 54 is sensing R-waves via electrodes 16. In this way, a clinician can be notified if an implantation needs to be adjusted in some way.

In some examples, when processing circuitry 50 detects that IMD 10 has been implanted, the processing circuitry may control optical emitter(s) 72 to adjust light color and/or brightness based on the skin color and/or skin tone of the patient. For example, either by viewing the IMD 10 through the skin or via the use of computing device 14, the clinician may observe the color and/or brightness of the optical emitter(s) following implantation. Through tapping detectable by accelerometer(s) 80 or via the computing device 14, the clinician may adjust the color and/or brightness of the light.

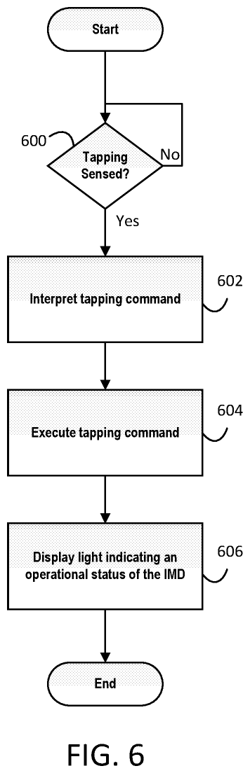

FIG. 6 is a flow chart illustrating another exemplary process for an IMD 10 according to some aspects of this disclosure.

Accelerometer(s) 80 may operate (e.g., in conjunction with processing circuitry 50) to determine whether tapping of the IMD 10 by a user is detected (600) and, in some cases, to determine a command associated with the tapping. For example, processing circuitry 50 may recognize an acceleration pattern by its correlation with a stored acceleration pattern corresponding to a tapping command. Any suitable tapping pattern may be used, such as a single tap, double tap, varying the timing between taps, etc. In some examples, different commands can be sent to the IMD 10 via different tapping patterns.

In response to detecting tapping (Yes of 600), processing circuitry 50 may accordingly interpret a detected tapping command (602). Based on the tapping command received, processing circuitry 50 may execute an instruction corresponding to the command (604), and optical emitter(s) 72 may display a light indicating an operational status of the IMD (606). In this manner, the acceleration sensor 70 can provide a mechanism to interface with the IMD 10, to send it commands, to change modes, etc. Tap detection may be operational before implantation and/or after implantation. For example, a clinician may tap the IMD 10 to request information from the IMD 10, such as whether a measured physiological parameter is at an acceptable quality. In another example, a patient may tap the IMD 10 to initiate a measurement or to initiate recording of a segment of heart activity.

The operational status of the IMD 10 that can be indicated by optical emitter(s) 72 is not limited to the examples described herein above. For example, the optical emitter(s) 72 may utilize a suitable blink timing or pattern, or color, or brightness, etc., to inform a patient of an ongoing measurement or to inform the patient that a battery is low. In another example, the optical emitter(s) 72 may be configured to blink/flash/shine with each R-wave detected by sensing circuitry 54 via electrodes 16. In another example, processing circuitry 50 controls optical emitter(s) 72 to provide an indicator when a cardiac arrest is detected based on the ECG sensed by sensing circuitry 54 via electrodes 16.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following are examples of the techniques described herein.

Example 1. An implantable medical device (IMD) comprising: a housing configured for subcutaneous implantation within a patient; a plurality of sensors on or within the housing, the plurality of sensors configured to sense one or more physiological parameters of the patient, wherein the plurality of sensors comprise an optical sensor comprising an optical emitter and an optical detector; and processing circuitry within the housing and configured to determine values of the one more physiological parameters using the plurality of sensors, wherein the processor is configured to: determine an operational status of the IMD; and cause the optical emitter to provide a visible indication of the operational status of the IMD.

Example 2. The IMD of example 1, wherein the operational status corresponds to at least one of the IMD being removed from packaging or ready for implantation.

Example 3. The IMD of example 2, wherein the processing circuitry is configured to determine that the IMD has been removed from packaging by at least detecting light with the optical detector.

Example 4. The IMD of example 2 or 3, wherein the plurality of sensors comprises a plurality of electrodes, wherein the processing circuitry is configured to determine that the IMD has been removed from the package by at least detecting a change in impedance via the electrodes.

Example 5. The IMD of any one or more of examples 1 to 4, further comprising communication circuitry within the housing, wherein the processing circuitry is configured to activate a communication mode of the communication circuitry based on determining that the IMD has been removed from packaging.

Example 6. The IMD of any one or more of examples 1 to 5, wherein the operational status corresponds to the IMD having been implanted into the patient.

Example 7. The IMD of example 6, wherein the processing circuitry is configured to determine that the IMD has been implanted into the patient using one or more of the plurality of sensors.

Example 8. The IMD of any one or more of examples 2 to 5, and 6 and 7, wherein the IMD is configured to activate the one or more of the plurality of sensors to determine that the IMD has been implanted in response to determining the at least one of the IMD being removed from packaging or ready for implantation.

Example 9. The IMD of any one or more of examples 6 to 8, further comprising communication circuitry within the housing, wherein the processing circuitry is configured to activate a communication mode of the communication circuitry based on determining that the IMD has been implanted within the patient.

Example 10. The IMD of any one or more of examples 1 to 9, wherein the operational status corresponds to a quality of measurement of at least one physiological parameter of the one or more physiological parameters by at least one sensor of the plurality of sensors.

Example 11. The IMD of example 10, wherein to determine the quality of measurement, the processing circuitry is configured to compare a metric of a signal from the at least one sensor of the plurality of sensors to a threshold.

Example 12. The IMD of any one or more of examples 1 to 11, wherein the operational status corresponds to the processing circuitry detecting at least one of a cardiac depolarization or a cardiac contraction via at least one sensor of the one or more sensors.

Example 13. The IMD of example 12, wherein the processing circuitry is configured to cause the optical emitter to provide a respective visible indication in response to detection of each of a plurality of cardiac depolarizations or cardiac contractions.

Example 14. The IMD of any one or more of examples 1 to 13, wherein the optical emitter is configured such that the visible indication is visible through a patient's skin after implantation of the IMD.

Example 15. The IMD of any one or more of examples 1 to 14, wherein the optical sensor is configured for at least one of photoplethysmography (PPG) or pulse oximetry.

Example 16. The IMD of any one or more of examples 1 to 15, wherein the plurality of sensors comprise an accelerometer, wherein the processing circuitry is configured to: detect a tap of the IMD by the user via the accelerometer; and activate the visible indication in response to detection of the tap.

Example 17. The IMD of any one or more of examples 1 to 16, wherein the IMD comprises an insertable cardiac monitor, wherein the housing has a length from a first end to a second end, a width, and a depth, wherein the length is greater than the width and the width is greater than the depth, wherein the length is within a range from 40 millimeters (mm) to 60 mm.

Example 18. The IMD of example 17, wherein the plurality of electrodes comprise a first electrode at or proximate to the first end, and a second electrode at or proximate to the second end, wherein the optical sensor is located between the first electrode and the second electrode along the length of the housing.

Example 19. A method of controlling an implantable medical device (IMD) to provide a visible indication to a user, the method comprising: determining, by processing circuitry of the IMD, an operational status of the IMD, wherein the IMD further comprises a housing configured for subcutaneous implantation within a patient and a plurality of sensors on or within the housing, the plurality of sensors configured to sense one or more physiological parameters of the patient, wherein the plurality of sensors comprise an optical sensor comprising an optical emitter and an optical detector; and causing, by the processing circuitry, the optical emitter to provide a visible indication of the operational status of the IMD.

Example 20. The method of example 19, wherein the operational status corresponds to at least one of the IMD being removed from packaging or ready for implantation.

Example 21. The method of example 20, wherein determining that the IMD has been removed from packaging comprises detecting light with the optical detector.

Example 22. The method of example 20 or 21, wherein the plurality of sensors comprises a plurality of electrodes, wherein determining that the IMD has been removed from packaging comprises detecting a change in impedance via the electrodes.

Example 23. The method of any one or more of examples 19 to 22, further comprising activating, by the processing circuitry, a communication mode of communication circuitry of the IMD based on determining that the IMD has been removed from packaging.

Example 24. The method of any one or more of examples 19 to 23, wherein the operational status corresponds to the IMD having been implanted into the patient.

Example 25. The method of example 24, wherein determining that the IMD has been implanted into the patient comprises using one or more of the plurality of sensors to determine that the IMD has been implanted into the patient.

Example 26. The method of any one or more of examples 20 to 23, and 24 and 25, further comprising, by the processing circuitry, activating the one or more of the plurality of sensors to determine that the IMD has been implanted in response to determining the at least one of the IMD being removed from packaging or ready for implantation.

Example 27. The method of any one or more of examples 24 to 26, further comprising activating, by the processing circuitry, a communication mode of communication circuitry of the IMD based on determining that the IMD has been implanted within the patient.

Example 28. The method of any one or more of examples 19 to 27, wherein the operational status corresponds to a quality of measurement of at least one physiological parameter of the one or more physiological parameters by at least one sensor of the plurality of sensors.

Example 29. The method of example 28, wherein determining the quality of measurement comprises comparing, by the processing circuitry, a metric of a signal from the at least one sensor of the plurality of sensors to a threshold.

Example 30. The method of any one or more of examples 19 to 29, wherein the operational status corresponds to the processing circuitry of the IMD detecting at least one of a cardiac depolarization or a cardiac contraction via at least one sensor of the one or more sensors.

Example 31. The method of example 30, wherein causing the optical emitter to provide the visible indication comprises causing the optical emitter to provide a respective visible indication in response to detection of each of a plurality of cardiac depolarizations or cardiac contractions.

Example 32. The method of any one or more of examples 19 to 31, wherein the optical emitter is configured such that the visible indication is visible through a patient's skin after implantation of the IMD.

Example 33. The method of any one or more of examples 19 to 32, wherein the optical sensor is configured for at least one of photoplethysmography (PPG) or pulse oximetry.

Example 34. The method of any one or more of examples 19 to 33, wherein the plurality of sensors comprise an accelerometer, the method comprising: detecting, by the processing circuitry, a tap of the IMD by the user via the accelerometer; and activating the visible indication in response to detection of the tap.

Example 35. A non-transitory computer readable storage medium comprising program instructions that cause processing circuitry of an implantable medical device (IMD) to perform a method comprising: determining an operational status of the IMD, wherein the IMD further comprises a housing configured for subcutaneous implantation within a patient and a plurality of sensors on or within the housing, the plurality of sensors configured to sense one or more physiological parameters of the patient, wherein the plurality of sensors comprise an optical sensor comprising an optical emitter and an optical detector; and causing the optical emitter to provide a visible indication of the operational status of the IMD.

1. An implantable medical device (IMD) comprising:
a housing configured for subcutaneous implantation within a patient;
a plurality of sensors on or within the housing, the plurality of sensors configured to sense one or more physiological parameters of the patient, wherein the plurality of sensors comprise an optical sensor comprising an optical emitter and an optical detector; and
processing circuitry within the housing and configured to determine values of the one more physiological parameters using the plurality of sensors, wherein the processing circuitry is configured to:
determine an operational status of the IMD; and
cause the optical emitter to provide a visible indication of the operational status of the IMD.

2. The IMD of claim 1, wherein the operational status corresponds to at least one of the IMD being removed from packaging or ready for implantation.

3. The IMD of claim 2, wherein the processing circuitry is configured to determine that the IMD has been removed from packaging by at least detecting light with the optical detector.

4. The IMD of claim 2, wherein the plurality of sensors comprises a plurality of electrodes, wherein the processing circuitry is configured to determine that the IMD has been removed from the packaging by at least detecting a change in impedance via the electrodes.

5. The IMD of claim 1, further comprising communication circuitry within the housing, wherein the processing circuitry is configured to activate a communication mode of the communication circuitry based on determining that the IMD has been removed from packaging.

6. The IMD of claim 1, wherein the operational status corresponds to the IMD having been implanted into the patient.

7. The IMD of claim 6, wherein the processing circuitry is configured to determine that the IMD has been implanted into the patient using one or more of the plurality of sensors.

8. The IMD of claim 7, wherein the IMD is configured to activate the one or more of the plurality of sensors to determine that the IMD has been implanted in response to determining at least one of the IMD being removed from packaging or ready for implantation.

9. The IMD of claim 6, further comprising communication circuitry within the housing, wherein the processing circuitry is configured to activate a communication mode of the communication circuitry based on determining that the IMD has been implanted within the patient.

10. The IMD of claim 1, wherein the operational status corresponds to a quality of measurement of at least one physiological parameter of the one or more physiological parameters by at least one sensor of the plurality of sensors.

11. The IMD of claim 10, wherein to determine the quality of measurement, the processing circuitry is configured to compare a metric of a signal from the at least one sensor of the plurality of sensors to a threshold.

12. The IMD of claim 1, wherein the operational status corresponds to the processing circuitry detecting at least one of a cardiac depolarization or a cardiac contraction via at least one sensor of the plurality of sensors.

13. The IMD of claim 12, wherein the processing circuitry is configured to cause the optical emitter to provide a respective visible indication in response to detection of each of a plurality of cardiac depolarizations or cardiac contractions.

14. The IMD of claim 1, wherein the optical emitter is configured such that the visible indication is visible through a patient's skin after implantation of the IMD.

15. The IMD of claim 1, wherein the optical sensor is configured for at least one of photoplethysmography (PPG) or pulse oximetry.

16. The IMD of claim 1,
wherein the plurality of sensors comprise an accelerometer,
wherein the processing circuitry is configured to:
detect a tap of the IMD by a user via the accelerometer; and
activate the visible indication in response to detection of the tap.

17. The IMD of claim 1, wherein the IMD comprises an insertable cardiac monitor,
wherein the housing has a length from a first end to a second end, a width, and a depth,
wherein the length is greater than the width and the width is greater than the depth, and
wherein the length is within a range from 40 millimeters (mm) to 60 mm.

18. The IMD of claim 17, wherein the plurality of electrodes comprises a first electrode at or proximate to the first end, and a second electrode at or proximate to the second end, wherein the optical sensor is located between the first electrode and the second electrode along the length of the housing.

19. A method of controlling an implantable medical device (IMD) to provide a visible indication to a user, the method comprising:
determining, by processing circuitry of the IMD, an operational status of the IMD, wherein the IMD further comprises a housing configured for subcutaneous implantation within a patient and a plurality of sensors on or within the housing, the plurality of sensors configured to sense one or more physiological parameters of the patient, wherein the plurality of sensors comprise an optical sensor comprising an optical emitter and an optical detector; and
causing, by the processing circuitry, the optical emitter to provide a visible indication of the operational status of the IMD.

20. The method of claim 19, wherein the operational status corresponds to at least one of the IMD being removed from packaging or ready for implantation.
